(19) United States
(12) Patent Application Publication
(10) Pub. No.: US 2009/0269387 A1
(43) Pub. Date: Oct. 29, 2009

(54) COMPOSITE IMPLANTS FOR PROMOTING BONE REGENERATION AND AUGMENTATION AND METHODS FOR THEIR PREPARATION AND USE

(76) Inventors: Yuval Zubery, Cochav Yair (IL); Arie Goldlust, Ness Ziona (IL); Thomas Bayer, Tel-Aviv (IL); Eran Nir, Rehovot (IL)

Correspondence Address: DANIEL J SWIRSKY, 55 REUVEN ST., BEIT SHEMESH, 99544, IL

(21) Appl. No.: 12/432,833

(22) Filed: Apr. 30, 2009

Related U.S. Application Data
(63) Continuation of application No. 11/829,111, filed on Jul. 27, 2007.
(60) Provisional application No. 60/833,476, filed on Jul. 27, 2006.

(51) Int. Cl. A61F 2/00; A61K 35/00; A61K 35/32; A61K 35/30; A61P 19/00

(52) U.S. Cl. 424/423; 424/93.7

(57) ABSTRACT

Collagen based matrices cross-linked by a reducing sugar(s) are used for preparing composite matrices, implants and scaffolds. The composite matrices may have at least two layers including reducing sugar cross-linked collagen matrices of different densities. The composite matrices may be used in bone regeneration and/or augmentation applications. Scaffolds including glycated and/or reducing sugar cross-linked collagen exhibit improved support for cell proliferation and/or growth and/or differentiation. The denser collagen matrix of the composite matrices may have a dual effect initially functioning as a cell barrier and later functioning as an ossification supporting layer. The composite matrices, implants and scaffolds may be prepared using different collagen types and collagen mixtures and by cross-linking the collagen(s) using a reducing sugar or a mixture of reducing sugars. The composite matrices, implants and scaffolds may include additives and/or living cells.

### CROSS-REFERENCE TO RELATED US APPLICATIONS

This application is a Continuation of U.S. patent application Ser. No. 11/829,111, filed on Jul. 27, 2007, which claims priority from and the benefit of U.S. Provisional Patent Application Ser. No. 60/833,476 filed on Jul. 27, 2006 entitled “Composite Implants for Promoting Bone Regeneration and Augmentation and Methods for Their Preparation and Use” incorporated herein by reference in its entirety.

### FIELD OF THE INVENTION

The present invention relates generally to implantable devices for promoting regeneration and augmentation of bone and more specifically of composite reducing sugar cross-linked collagen based matrices, methods for their use and methods for their preparation.

### BACKGROUND OF THE INVENTION

Alveolar bone loss is secondary to early tooth loss and periodontal disease, leading to severe functional and esthetic problems. In the last three decades the replacement of missing or hopeless teeth is possible via the use of dental implants. These, however require sufficient bony housing to accommodate an implant of appropriate length and diameter to be able to withstand the oclussal load on the future prosthetic device, and to provide optimal esthetic results. Thus, in many cases, alveolar bone augmentation is mandatory for functional and esthetic long term success of dental implants.

The most common techniques for bone augmentation procedures involve the use of bone grafts under a barrier that prevents soft tissue invasion, and allows a selective cell line with osteogenic capabilities to populate the defect. These are used to facilitate migration and differentiation of mesenchymal cells to form osteoblasts and lay down bone within the defect. In addition, such devices may serve as a scaffold that supports cell migration. The grafts may be derived from natural sources (human and other animals), or from various synthetic materials, as is known in the art. Bone grafts are normally used as a powder with particle size ranging from 0.25-2 mm mixed with patient's blood as a coagulum or mixed with sterile saline. In some cases, gel or putty like consistency of the implant provide improved handling of the material.

A major shortcoming of such bone grafts is the long term resorption and replacement of the graft that may compromise the mechanical properties of the resulting augmented bone.

Similar problems may also be encountered in the treatment of various bone defects such as orthopaedic bone deficiencies. These devices (matrices) may be used for augmentation and treatment of bone fractures, and the like.

Materials for supporting bone augmentation should ideally have the following properties:
1. The ability to mechanically support a barrier.
2. The graft material should be biocompatible with minimal allergic or immunogenic reactions.
3. The graft should be safe from risk of disease transmission.
4. The graft material should preferably serve as a scaffold that encourages cells to migrate and populate the secluded space of the bone defect.
5. The graft should preferably undergo complete degradation within 6-12 months.
6. The graft should preferably mimic bone matrix proteins and should be capable of undergoing ossification.
7. Preferably the graft should serve as a carrier for suitable growth factors.
8. The graft should be easy to handle even by inexperienced clinicians requiring minimal skills for its preparation and implantation to save time and reduce possible complications.

It would therefore be advantageous to have a bone graft or implant combining as many as possible of the above properties.

### SUMMARY OF THE INVENTION

There is therefore provided, in accordance with an embodiment of a method of the present application a method for preparing a composite multi-density cross-linked collagen implantable device. The method includes the steps of, compressing a suspension including fibrillated collagen particles in a first suspending solution to form a first matrix having a first density, applying to the first matrix a suspension including fibrillated collagen particles in a second suspending solution to form a second matrix attached to the first matrix the second matrix having a second density lower than the first density, drying the first matrix and the second matrix to form a dry multi-density composite matrix, and reacting the multi-density composite matrix with a reducing sugar to form the composite multi-density cross-linked collagen implantable device.

Furthermore, in accordance with an embodiment of the method of the present application, the step of reacting includes incubating the composite multi-density implantable device with a reducing sugar in an incubation solution including ethanol.

Furthermore, in accordance with an embodiment of the method of the present application, the incubation solution includes 70% ethanol.

Furthermore, in accordance with an embodiment of the method of the present application, the reducing sugar is selected from D(-) ribose and DL glyceraldehyde.

Furthermore, in accordance with an embodiment of the method of the present application, at least one additional substance is added to at least one of the first suspending solution, said second suspension solution, said first matrix, and said second matrix.

Furthermore, in accordance with an embodiment of the method of the present application, the method also includes the step of adding living cells to the composite implantable device. The cells are selected from cultured cells, stem cells, human cells, animal cells, fibroblasts, pluripotent bone marrow cells, pluripotent stem cells, bone building cells, osteoblasts, mesenchymal cells, mammalian cells, primary cells, genetically modified cells, nerve cells and any combinations thereof.

There is also provided, in accordance with an embodiment of the implantable device of the present application, a composite multi-density cross-linked collagen implantable device prepared by any of the above methods.

There is also provided, in accordance with an embodiment of the implants of the present application, a composite multi-density cross-linked collagen based implant. The implant includes a first reducing sugar cross-linked collagen based matrix having a first density and at least a second reducing sugar cross-linked collagen based matrix attached to the first reducing sugar cross-linked collagen based matrix. The second collagen based matrix has a second density lower than the first density.

Furthermore, in accordance with an embodiment of the implants of the present application, the first and the second reducing sugar cross-linked collagen based matrices are obtained by cross-linking collagen with a reducing sugar in an incubation solution including ethanol.

Furthermore, in accordance with an embodiment of the implants of the present application, the incubation solution comprises 70% ethanol.

Furthermore, in accordance with an embodiment of the implants of the present application, the reducing sugar is selected from D(-) ribose and DL glyceraldehyde.

Furthermore, in accordance with an embodiment of the implants of the present application, the composite implant includes at least one additional substance.

Furthermore, in accordance with an embodiment of the implants of the present application, the implant includes living cells selected from cultured cells, stem cells, human cells, animal cells, fibroblasts, pluripotent bone marrow cells, pluripotent stem cells, bone building cells, osteoblasts, mesenchymal cells, mammalian cells, primary cells, genetically modified cells, nerve cells and any combinations thereof.

There is also provided, in accordance with an embodiment of the methods of the present application, a method for using a composite multi-density cross-linked collagen implantable device for treating a bone defect. The method includes the step of applying to the bone defect a composite multi-density glycated cross-linked collagen based implantable device including a first reducing sugar cross-linked collagen based matrix having a first density and at least a second reducing sugar cross-linked collagen based matrix attached to the first collagen based matrix. The second collagen based matrix has a second density lower than the first density. The at least second collagen based matrix is disposed within the bone defect to promote bone formation within the bone defect. The first collagen based matrix at least partially prevents the formation of tissue other then bone tissue within the bone defect.

Furthermore, in accordance with an embodiment of the methods of the present application, the implantable device is obtained by incubating a collagen based composite multi-density implantable device with a reducing sugar in an incubation solution including ethanol.

Furthermore, in accordance with an embodiment of the methods of the present application, the incubation solution includes 70% ethanol.

Furthermore, in accordance with an embodiment of the methods of the present application, the reducing sugar is selected from D(-) ribose and DL glyceraldehyde.

Furthermore, in accordance with an embodiment of the methods of the present application, the composite implantable device includes least one additional substance.

There is also provided, in accordance with an embodiment of the methods of the present application, a method for using a reducing sugar cross-linked collagen matrix as an improved scaffold for cell proliferation and cell differentiation. The method includes the steps of providing a scaffold comprising a collagen matrix cross-linked with a reducing sugar, and incubating the scaffold with living cells to induce improved growth and/or proliferation and/or differentiation of the cells.

Furthermore, in accordance with an embodiment of the methods of the present application, the cells are selected from cultured cells, stem cells, human cells, animal cells, fibroblasts, pluripotent bone marrow cells, pluripotent stem cells, bone building cells, osteoblasts, mesenchymal cells, mammalian cells, primary cells, genetically modified cells, nerve cells and any combinations thereof.

Furthermore, in accordance with an embodiment of the methods of the present application, the scaffold is obtained by incubating a collagen based matrix with a reducing sugar in an incubation solution including ethanol.

Furthermore, in accordance with an embodiment of the methods of the present application, the incubation solution includes 70% ethanol.

Furthermore, in accordance with an embodiment of the methods of the present application, the reducing sugar is selected from D(-) ribose and DL glyceraldehyde.

Furthermore, in accordance with an embodiment of the methods of the present application, the scaffold comprises at least one additional substance.

Finally, in accordance with additional embodiments of the methods, scaffolds, composite matrices and composite implants of the present application, the at least one additional substance is selected from an antimicrobial agent, an anti-inflammatory agent, an anti-bacterial agent, an anti-fungal agent, one or more factors having tissue inductive properties, growth factors, growth promoting and/or growth inhibiting proteins or factors, extracellular matrix components, an anesthetic material, an analgesic material, an osteoblast attracting factor, a drug, a pharmaceutical agent, a pharmaceutical composition, a protein, a glycoprotein, a mucoprotein, a mucopolysaccharide, a glycosaminoglycan, hyaluronic acid, chondroitin 4-sulfate, chondroitin 6-sulfate, keratan sulfate, dermatan sulfate, heparin, heparan sulfate, a proteoglycan, a lecitin rich interstitial proteoglycan, decorin, biglycan, fibromodulin, lumican, aggrecan, syndecans, beta-glycan, versican, centroglycan, serglycin, fibronectins, fibroglycan, chondroadherins, fibulins, thrombospondin-5, calcium phosphate, hydroxyapatite, alkaline phosphatase, pyrophosphatase, a material related to gene therapy, DNA, RNA, a fragment of DNA or RNA, a nucleic acid, an oligonucleotide, a polynucleotide, a plasmid, a vector, an allogeneic material, a nucleic acid, an oligonucleotide, a chimeric DNA/RNA construct, a DNA probe, an RNA probe, anti-sense DNA, anti-sense RNA, a gene, a part of a gene, a composition including naturally or artificially produced oligonucleotides, a plasmid DNA, a cosmid DNA, a viral genetic construct, hyaluronan, a hyaluronan derivative, a hyaluronan salt a hyaluronan ester, chitosan, a chitosan derivative, a chitosan salt, a chitosan ester thereof, an oligosaccharide, a polysaccharides, a polysaccharides salt, a polysaccharides derivative, a polysaccharides ester, an oligosaccharide derivative, an oligosaccharide salt, an oligosaccharide ester, a biocompatible synthetic polymer, a cross-linked protein, a cross-linked glycoprotein, a non-cross-linked glycoprotein, calcium phosphate nanoparticles, hydroxy-apatite crystals, a growth factors, a BMP, PDGF and any combinations thereof.

### BRIEF DESCRIPTION OF THE DRAWINGS

In order to understand the invention and understand how it may be carried out in practice, several preferred embodiments will now be described, by way of non-limiting example only, with reference to the accompanying drawings:

FIG. 1 is a composite photomicrograph representing several regions of tissue excised from an implant of a rat calvarial experimental bone defect twelve weeks after the implantation of a composite matrix comprising a scaffold including a reducing sugar cross-linked collagen based sponge and a reducing sugar cross-linked collagen barrier membrane;

FIG. 2 is a schematic cross-sectional view representing a composite implantable cross-linked collagen matrix having parts with different densities in accordance with an embodiment of the method of the present invention;

FIG. 3 is a photograph representing a composite implantable cross-linked collagen matrix having parts with different densities prepared from porcine collagen for treating bone defects, in accordance with an embodiment of a method of the present invention;

FIG. 4 is a schematic graph representing a schematic cross sectional view of a bone defect treated with an implantable composite cross-linked collagen matrix having parts with different densities for treating bone defects, in accordance with an embodiment of a method of the present invention; and

FIG. 5 is a schematic graph representing the results of an in-vitro experiment quantitatively comparing the fibroblast population of a collagen sponge based on ribose cross-linked porcine collagen with the fibroblast population of another commercially available collagen sponge based on collagen stabilized with formaldehyde.

### DETAILED DESCRIPTION OF THE INVENTION

It is noted that for the purposes of the present application the term “reducing sugar” is defined as any natural and/or artificial reducing sugar and any derivatives of such reducing sugars, including but not limited to, glycerose (glyceraldehyde), threose, erythrose, lyxose, xylose, arabinose, allose, altose, glucose, manose, gulose, idose, galactose, fructose, talose, a diose, a triose, a tetrose, a pentose, a hexose, a septose, an octose, a nanose, a decose, a reducing disaccharide, maltose, lactose, cellobiose, gentiobiose, melibiose, turanose, trehalose and a reducing trisaccharide and a reducing oligosaccharide, and any derivatives of such reducing sugars.

The term collagen is defined for the purposes of the present application as any form of natural collage and/or purified collagen and/or chemically modified collagen, and/or proteolitically treated collagen, and/or genetically engineered collagen, and/or artificially produced collagen, including but not limited to, native collagen, fibrillar collagen, fibrillar atelopeptide collagen, lyophylized collagen, freeze dried collagen, collagen obtained from animal sources, a collagen produced by a genetically modified plant and/or microorganism and/or mammal and/or multicellular organism, porcine collagen, bovine collagen, human collagen, recombinant collagen, pepsinized collagen, reconstituted collagen, reconstituted purified collagen, reconstituted attelopeptyde purified collagen, and any combinations thereof.

Experiment 1

This experiment describes histological evidence of new bone formation in vivo within collagen matrices cross-linked with a reducing sugar. A rat calvarial model was used to study the performance of a collagen based sponge-like matrix material cross-linked with a reducing sugar as an ossification promoting bone defect filler material useable in association with a collagen based membrane barrier.

Critical size defects (5 mm diameter) were surgically created in the skull of young rats, as described in a paper by Verna et al. (Verna C, Bosch C, Dalstra M, et al. Healing patterns in calvarial bone defects following guided bone regeneration in rats. J. Clin. Periodontol. 2002; 29:865-870) incorporated herein by reference in its entirety.

The bone defects were filled with a trimmed to fit ribose cross-linked porcine collagen sponge (prepared as described hereinafter-see for example EXAMPLE 4 below) and covered with trimmed Ossix™—PLUS glycated collagen barrier membrane, commercially available from ColBar LifeScience Ltd., Herzliya, Israel. At four, eight and twelve weeks after implantation, the rats were sacrificed and the implanted sites were excised. Paraffin blocks of the excised implants were created and serial sections were cut and stained with Mallory Trichrome stain.

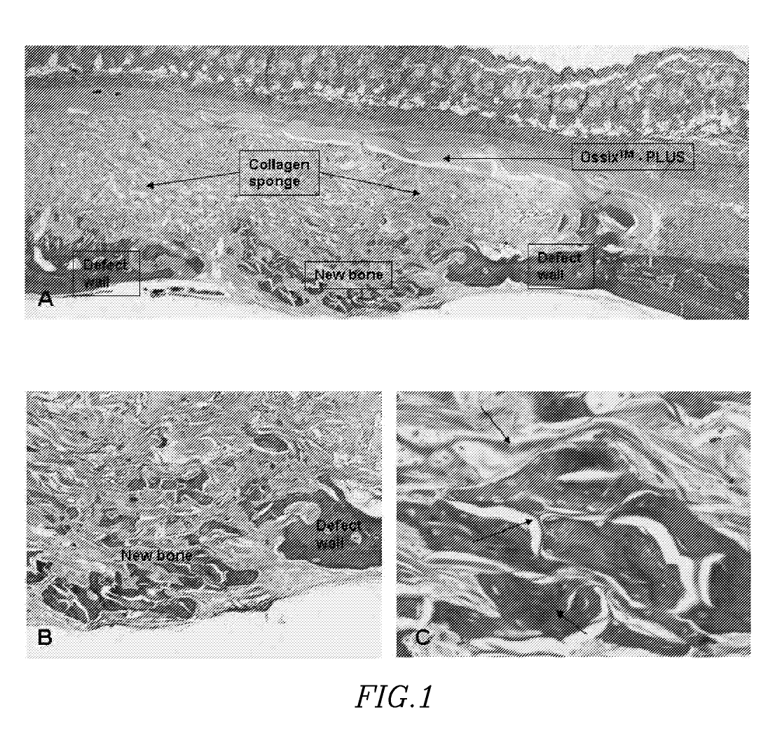

At twelve weeks after implantation, distinct areas of newly formed bone were noticed within the sponge under microscope visual examination of the serial sections. The newly formed bone created a bridge from one side of the defect to the other, suggesting the capability of the sponge to act as a biological scaffold enabling complete resolution of the defect. Moreover, new bone formed within the sponge above the original envelope of bone suggesting that the sponge may be able to augment bone. The histological results are presented in FIG. 1.

The barrier effect provided by the Ossix™—PLUS membrane (preventing the fast growing fibroblasts from populating the sponge) supports the observed bone augmentation since without its presence (sponge alone, data not shown) no new bone formation was observed.

Reference is now made to FIG. 1 which is a composite photomicrograph, representing cross-sections of tissue excised from rat calvarial bone defect experimental model at 12 weeks after treatment with a combination of a collagen sponge and barrier membrane as described hereinabove (stained with Mallory Trichrome stain).

In the micrograph labeled A of FIG. 1, newly formed bone bridging the defect may be observed within the sponge. Residues of the Ossix™—PLUS barrier membrane lie above the sponge. (original magnification ×4).

The micrograph labeled B of FIG. 1 represents a higher magnification of defect area (original magnification ×10). Note areas in which new bone is formed within the sponge above the original envelope of bone.

The micrograph labeled C of FIG. 1 represents a different magnified area (original magnification ×40) from the photomicrograph of the part labeled A of FIG. 1. New bone is formed within the sponge's cavities and the walls of the sponge may be observed (arrows).

The results of the experiments described hereinabove demonstrate substantial bone augmentation inside the collagen sponge material when used in association with a collagen based membrane barrier. It is interesting to note here that at the twelve week model animal group, there was substantial and clearly observable bone augmentation in the sponge-like (lower density) area. The collagen barrier membrane showed signs of mineralization which may represent the first step in the ossification of the denser Ossix™—PLUS barrier membrane which was used to cover the sponge.

Additional in-vivo experiments in dogs supporting the novel superior bone regenerating and bone augmentation properties of the sugar cross-linked collagen matrices of the present application are disclosed in the article entitled “OSSIFICATION OF A NOVEL CROSS-LINKED PORCINE COLLAGEN BARRIER FOR GUIDED BONE REGENERATION IN DOGS” by Yuval Zubery, Arie Goldlust, Antoine Alves, and Eran Nir, published in Journal of Periodontology 78, 112-121 (2007), incorporated herein by reference in its entirety. The results of these experiments further support the novel and unexpected superior properties of the porcine ribose cross-linked collagen matrices in promoting bone regeneration and bone augmentation in comparison with other commercially available collagen membranes which were cross-linked with other different cross-linkers, as described in detail in the article.

It is noted that the dual, time dependent, effect of the denser barrier membrane was also clearly demonstrated in the above mentioned article by Zubery et al. which clearly shows that while initially the denser barrier membrane functions as an effective barrier preventing the penetration of fibroblasts into the bone defect region occupied by the less dense collagen sponge layer, at a later stage of the defect healing process, bone forming cells successfully invade the denser collage barrier membrane resulting in substantially complete ossification of the barrier membrane and participating in improving the bone regeneration and augmentation process.

In accordance with another embodiment of the present invention there is provided a composite bone graft implant that includes a part with a relatively low density of collagen based material serving as a scaffold for bone regeneration and augmentation and another part having higher density of collagen for initially serving as a barrier for preventing invasion of other non-bone forming cells and tissue into the bone defect. An unexpected advantage of the composite bone graft is that while the barrier (higher density part) of the composite implant initially functions as a barrier material, it also supports further ossification of the defect at later stages of the augmentation process by being itself ossified.

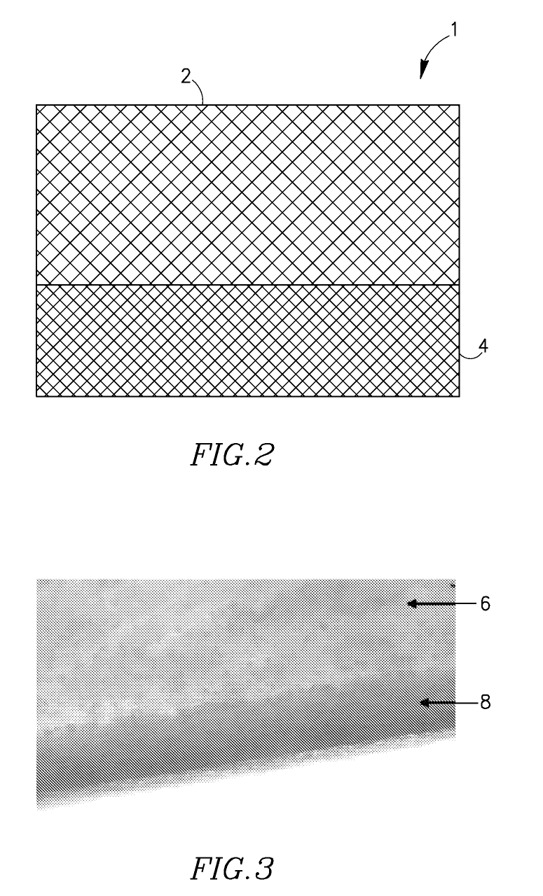

Reference is now made to FIG. 2 which is a schematic cross-sectional view representing a composite implantable cross-linked collagen matrix having parts with different densities in accordance with an embodiment of the method of the present invention. The composite matrix 1 includes a first portion 2 which includes reducing sugar cross-linked collagen having a relatively low density (sponge-like structure) conducive to bone forming cells or tissues and serving as a scaffold for bone tissue formation therein. The composite matrix 1 also includes a second portion 4 which includes reducing sugar cross-linked collagen having a relatively high density which may act (at least initially) as a barrier for preventing or reducing the penetration of unwanted cells or tissues into the first portion 2 of the matrix 1 to reduce or prevent the formation of connective tissue in the first portion 2 of the matrix. An advantage of the composite matrix is that the portion 4 in addition to serving as a barrier as explained hereinabove may also enhance bone augmentation by supporting (at least in the more advanced stages of the augmentation) bone formation by being ossified.

### EXAMPLE 1

Porcine fibrillar collagen was prepared as described in detail in the U.S. Pat. No. 6,682,760, incorporated herein by reference in its entirety. The fibrillated collagen was concentrated by centrifugation at 4500 rpm. All centrifugations (unless specifically stated otherwise) were done using a model RC5C centrifuge with a SORVALL SS-34 rotor commercially available from SORVALL® Instruments DUPONT, USA.

The fibrillated collagen concentration after centrifugation was 75 mg/mL (as determined by Lowry standard method).

50 milliliters (50 mL) fibrillated collagen were poured into a 140 mm×120 mm stainless steel tray. The fibrillated collagen was equally dispersed and covered with a mesh (Propyltex 05-1 25/30, commercially available from SEFAR AG, Heiden, Switzerland), A perforated polystyrene plate was placed on top of the mesh and a 5 kilogram weight was placed on top of the plate in order to compress the fibrillated collagen. The compression lasted for 18 hours at 4° C. After the compression, the weight was removed, the released buffer solution was drained and the mesh was removed to yield a first portion of compressed fibrillated collagen. 100 mL of a suspension of fibrillated collagen (37.5 mg/mL) in 10 millimolar phosphate buffer solution (PBS pH 7.36) were poured and evenly distributed on top of the compressed, fibrillated collagen layer. The tray was transferred into the lyophilizer (Freeze dryer model FD 8 commercially available from Heto Lab Equipment DK-3450 Allerod, Denmark), pre-frozen for eight hours and lyophilized for 24 hours. The condenser temperature was −80° C. The shelf temperature during pre-freezing was −40° C. The shelf temperature during lyophilization: was +30° C. and the vacuum during lyophilization was approximately 0.01 bar.

200 mL of a solution containing 120 mL absolute ethanol, 80 mL PBS buffer solution (10 mM, pH 7.36) and 2 gram of DL-glyceraldehyde was added to the dried fibrillated collagen and incubated at 37° C. for 24 hours to perform the cross-linking of the composite collagen structure. Afterwards, the combined collagen product was washed exhaustively with DI water and lyophilized, using the same conditions as described above.

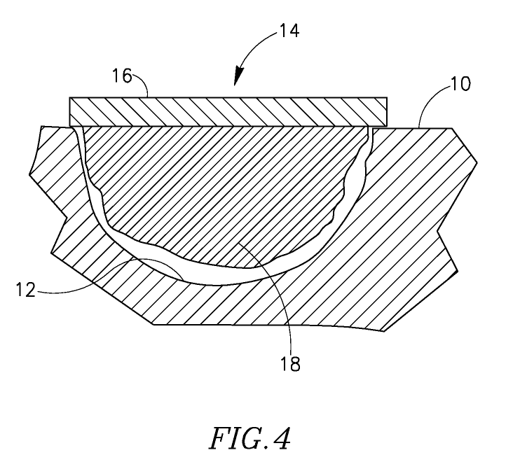

Reference is now made to FIG. 3 which is a photograph representing a composite implantable cross-linked collagen matrix having parts with different densities prepared from porcine collagen for treating bone defects, in accordance with an embodiment of a method of the present invention as described hereinabove in EXAMPLE 1. The region labeled 6 represents the lower density portion of the composite matrix and the region labeled 8 represents the denser portion which functions as a barrier layer.

### EXAMPLE 2

Porcine fibrillar collagen was prepared as described in detail in the U.S. Pat. No. 6,682,760, incorporated herein by reference in its entirety. The fibrillated collagen was concentrated by centrifugation at 4500 rpm. All centrifugations (unless specifically stated otherwise) were done using a model RC5C centrifuge with a SORVALL SS-34 rotor commercially available from SORVALL® Instruments DUPONT, USA.

450 mL of purified collagen (concentration: 2.73 mg/mL) were mixed with 50 mL fibrillation buffer (as described in detail in the U.S. Pat. No. 6,682,760) and poured into a tray. The mixture was incubated for 18 hour at 37° C. to form a gel. The fibrillated collagen was covered with a mesh (Propyltex 05-1 25/30, commercially available from SEFAR AG, Heiden, Switzerland), A perforated stainless steel plate was placed on top of the mesh and a 1.9 kg weight was placed on the gel for 18 hours at 37° C. to compress the gel to form a membrane.

After the compression, the weight was removed, the released buffer solution was drained and the mesh was removed to yield a first portion of compressed fibrillated collagen. The compressed membrane was placed in a 140 mm×120 mm stainless steel tray and 100 mL of a suspension of porcine fibrillated collagen (37.5 mg/mL) in 10 millimolar phosphate buffer solution (PBS pH 7.36) prepared as described in detail in the U.S. Pat. No. 6,682,760, were poured and evenly distributed on top of the compressed, fibrillated collagen layer. The tray was transferred into the lyophilizer (Freeze dryer model FD 8 commercially available from Heto Lab Equipment DK-3450 Allerod, Denmark), pre-frozen for eight hours and lyophilized for 24 hours. The condenser temperature was −80° C. The shelf temperature during pre-freezing was −40° C. The shelf temperature during lyophilization was +30° C. and the vacuum during lyophilization was approximately 0.01 bar.

200 mL of a solution containing 120 mL absolute ethanol ( commercially available from Merck, Germany), 80 mL PBS buffer solution (10 mM, pH 7.36) and 2 gram of DL-glyceraldehyde (commercially available as Catalogue No. G5001 from Sigma, USA) were added to the dried (lyophilized) fibrillated collagen and incubated at 37° C. for 24 hours to perform the cross-linking of the composite collagen structure. The combined collagen product was washed exhaustively with DI water and lyophilized, using the same conditions as described above.

### EXAMPLE 3

Porcine fibrillar collagen was prepared as described in detail in the U.S. Pat. No. 6,682,760 incorporated herein by reference in its entirety. The fibrillated collagen was concentrated by centrifugation at 4500 rpm. All centrifugations (unless specifically stated otherwise) were done using a model RC5C centrifuge with a SORVALL SS-34 rotor commercially available from SORVALL® Instruments DUPONT, USA.

450 mL of purified collagen (concentration: 2.73 mg/mL) were mixed with 50 mL fibrillation buffer (as described in detail in the U.S. Pat. No. 6,682,760) and poured into a tray. The mixture was incubated for 18 hour at 37° C. to form a gel. The fibrillated collagen was covered with a mesh (Propyltex 05-1 25/30, commercially available from SEFAR AG, Heiden, Switzerland), A perforated stainless steel plate was placed on top of the mesh and a 1.9 kg weight was placed on the gel for 18 hours at 37° C. to compress the gel to form a membrane.

After the compression, the weight was removed, the released buffer solution was drained and the mesh was removed to yield a first portion of compressed fibrillated collagen. The compressed membrane was placed in a 140 mm×120 mm stainless steel tray and 100 mL of a suspension of porcine fibrillated collagen (37.5 mg/mL) in 10 millimolar phosphate buffer solution (PBS pH 7.36) prepared as described in detail in the U.S. Pat. No. 6,682,760 were poured and evenly distributed on top of the compressed, fibrillated collagen layer. The tray was transferred into the lyophilizer (Freeze dryer model FD 8 commercially available from Heto Lab Equipment DK-3450 Allerod, Denmark), pre-frozen for eight hours and lyophilized for 24 hours. The condenser temperature was −80° C. The shelf temperature during pre-freezing was −40° C. The shelf temperature during lyophilization was +30° C. and the vacuum during lyophilization was approximately 0.01 bar.

200 mL of a solution containing 120 mL absolute ethanol (commercially available from Merck, Germany), 80 mL PBS buffer solution (10 mM, pH 7.36) and 3 gram of D(-)Ribose (commercially available as Catalogue No. R7500 from Sigma, USA) were added to the dried (lyophilized) fibrillated collagen and incubated at 37° C. for 14 days to perform the ribose cross-linking of the composite collagen structure. The ribose cross-linked combined collagen product was washed exhaustively with DI water and lyophilized, using the same conditions as described above.

### EXAMPLE 4

Porcine fibrillar collagen was prepared as described in detail in the U.S. Pat. No. 6,682,760 incorporated herein by reference in its entirety. The fibrillated collagen was concentrated by centrifugation at 4500 rpm. All centrifugations (unless specifically stated otherwise) were done using a model RC5C centrifuge with a SORVALL SS-34 rotor commercially available from SORVALL® Instruments DUPONT, USA.

The fibrillated collagen concentration after centrifugation was 15 mg/mL (as determined by Lowry standard method).

100 mL of a suspension of porcine fibrillated collagen (15.0 mg/mL) in 10 millimolar phosphate buffer solution (PBS pH 7.36) prepared as described in detail in the U.S. Pat. No. 6,682,760, were poured into a stainless steel tray. The tray was transferred into the lyophilizer (Freeze dryer model FD 8 commercially available from Heto Lab Equipment DK-3450 Allerod, Denmark), pre-frozen for eight hours and lyophilized for 24 hours. The condenser temperature was −80° C. The shelf temperature during pre-freezing was −40° C. The shelf temperature during lyophilization was +30° C. and the vacuum during lyophilization was approximately 0.01 bar.

200 mL of a solution containing 120 mL absolute ethanol (commercially available from Merck, Germany), 80 mL PBS buffer solution (10 mM, pH 7.36) and 3 gram of D(-) ribose (commercially available as Catalogue No. R7500 from Sigma, USA) were added to the dried (lyophilized) fibrillated collagen and incubated at 37° C. for 4, 7, 11 and 14 days to perform the ribose cross-linking of the collagen structure. The ribose cross-linked collagen products were washed exhaustively with DI water and lyophilized, using the same conditions as described above.

The advantage of using such a composite matrix as described hereinabove in Examples 1-3 and illustrated in FIGS. 2 and 3, is that it is not necessary to prepare and shape two different types of devices as was done in the rat model experiments described above. Rather, the physician, surgeon, or dentist using the composite matrix may simply cut a piece of the material 1 to a size and shape approximating the size and shape of the bone defect and may further trim the cut piece as necessary after checking it against the defect.

After the necessary shape and size have been achieved, the user or physician inserts the shaped matrix into the defect in the bone with the low density portion 6 filling the defect and the denser barrier portion 8 being positioned (see FIG. 4 Below) to face the tissues or environment outside the treated bone defect.

Reference is now made to FIG. 4 which is a cross-sectional diagram illustrating a cross section of a bone defect treated with a implantable composite cross-linked collagen matrix 16 having parts with different densities for treating bone defects, in accordance with an embodiment of a method of the present invention. The bone 10 has a bone defect 12 therein. The shaped composite matrix 14 is inserted into the defect 12 so that the portion 18 having the lower density faces the walls of the defect 12 and the denser barrier portion 16 is positioned adjacent the surface of the bone 10, preferably entirely covering the opening of the defect 12 to prevent penetration of unwanted cells (such as, for example, fibroblasts) populating the space of the defect 12 and/or the lower density portion 18 of the composite matrix 14. The portion 18 may thus function as a suitable ossification substrate (scaffold) for bone tissue growth while being protected by the portion 16 of the composite matrix 14 which functions as a barrier preventing or reducing the penetration of fibroblasts and/or other undesirable cells or tissues into the defect 12 and/or into the portion 18.

As bone building advances within the portion 18 and the defect 12 gets filled with bone tissue, the portion 16 may gradually ossify as well, enhancing bone augmentation and the integrity of the augmented bone tissue.

In-vitro Cell Growth Experiments with a Reducing Sugar Cross-Linked Collagen Sponge

The possibility of growing tissue within the sponge was also evaluated in vitro through cell culture of different cell types. Primary cultured human foreskin fibroblasts as well as pluripotent mouse bone marrow cell line (D1) penetrated the reducing sugar cross-linked sponge and proliferated very well within the sponge cavities.

Experiment 2

Ribose cross-linked collagen porcine sponge was prepared as disclosed hereinabove in EXAMPLE 4). The glycation (and cross-linking) incubation was performed at 37° C. for seven days to perform the ribose cross-linking of the collagen structure. The ribose cross-linked collagen products were washed exhaustively with DI water and lyophilized, using the same conditions as described above. The ability of the resulting ribose cross-linked collagen sponge to serve as a scaffold for support proliferation and/or differentiation of human foreskin fibroblasts was compared to bovine collagen sponge product (CollaCot®) commercially available from Sulzer Medica (Sulzer Dental Inc. USA). It is noted that as Sulzer Dental Inc. was recently bought by Zimmer Dental Inc., CA, U.S.A the same sponge product under the same name CollaCot® continues to be commercially available from Zimmer Dental Inc., CA, U.S.A.

The Sulzer CollaCot® sponge includes bovine collagen extracted from bovine deep flexor (Achilles) tendon and GAG, and stabilized with formaldehyde.

Small pieces of the resulting cross-linked collagen sponge were incubated with primary cultured human foreskin fibroblasts. Primary fibroblasts (from human foreskin) of passage 16 were used. Two 100 mL cell spinners equipped with a rotating basket were used for seeding the sponges. The Sponges were placed in the basket (6 sponges per spinner) and seeded with fibroblasts. In the first Spinner, six of the Colbar (ribose cross-linked porcine collagen) sponges were seeded with 71×10⁶ fibroblast cells. In the second Spinner, six of the commercial Sulzer CollaCot® sponge (formaldehyde stabilized bovine collagen) sponges were seeded with 79×10⁶ of the same fibroblast cells.

DMEM (Dulbeco Modified Eagle's Medium) Grow medium supplemented with 20 mM HEPES (4-(2-hydroxyethyl)-1-piperazineethanesulfonic acid), 10% FBS (Fetal bovine serum) and 20 mg/mL Gentamycin was used throughout the entire experiment. After seeding the sponges were incubated in a tissue culture incubator at 37° C., with medium changes performed approximately every two days. The cell populated sponges were harvested at twenty (20) days after seeding and histology and quantitative analysis was performed.

The sponge was then removed, fixed and embedded in paraffin for crio-sectioning using standard techniques. 5 μm thick paraffin sections of the sponge were stained with Hematoxylin & Eosine stain. The stained sections were microscopically observed at magnifications of ×10-×40 active primary human fibroblasts were observed to produce a loose network of new collagen within the sponge cavities. These newly formed collagen networks were in contact with other fibroblasts as well as with the sponge collagen walls.

Visual examination of the photomicrographs revealed that primary cultured human fibroblasts proliferate in the ribose-cross-linked porcine collagen sponge homogenously. In contrast, the same fibroblasts grow (to a much lesser extent) primarily at the edges of Sulzer Medica's bovine collagen sponge and not in the middle section of the sponge possibly indicating greater difficulty of cell penetration of and migration into the Sulzer Medica's sponge.

The microscopic observation of loose collagen formation by the human foreskin fibroblasts and their ability to form an epithelial like layer on the edges of the sponge implies that the COLBAR ribose cross-linked collagen sponge (also referred to as the COLBAR sponge hereinafter) may be a favorable scaffold for the proliferation and differentiation of tissue. The growth of human fibroblasts within the glycated and cross-linked collagen sponge was also compared with a commercially available bovine collagen sponge (CollaCote®) and was unexpectedly found to be superior in the COLBAR sponge. Pluripotent stem cells also flourished within the sponge suggesting the possibility of inducing differentiation while using the COLBAR reducing sugar cross-linked collagen sponge as a biological scaffold.

A quantitative evaluation of the degree of fibroblast distribution within the two different sponges was also conducted. serial paraffin sections were taken from paraffin embedded blocks of the porcine ribose cross linked collagen sponge and the Sulzer bovine formaldehyde stabilized collagen sponge. For each sponge ten microtome serial sections, each having a thickness of six micron, were cut and only every third section was analyzed (such that there was a 12 micron spacing between the analyzed sections). Sections No. 1, 4, 7 and 10 (i.e. the first, fourth, seventh and tenth sections) of each sponge were analyzed by an automatic cell counting technique. These four sections represented a 60 micrometer deep rectangular portion for each sponge.

The automatic cell counting was performed using a Nikon Eclipse 50i microscope with a Maerzhauser Scan 100×80 Motorized microscope stage. The microscope was coupled to a Nikon Digital Sight DS-5M Camera. The Lens magnification was 10×. A stitched image composed of multiple images spanning the whole length of the sponge was formed by using the NIS Elements AR 2.30 SP4 Build 384 software commercially available from Nikon Instruments Inc., NY, U.S.A.

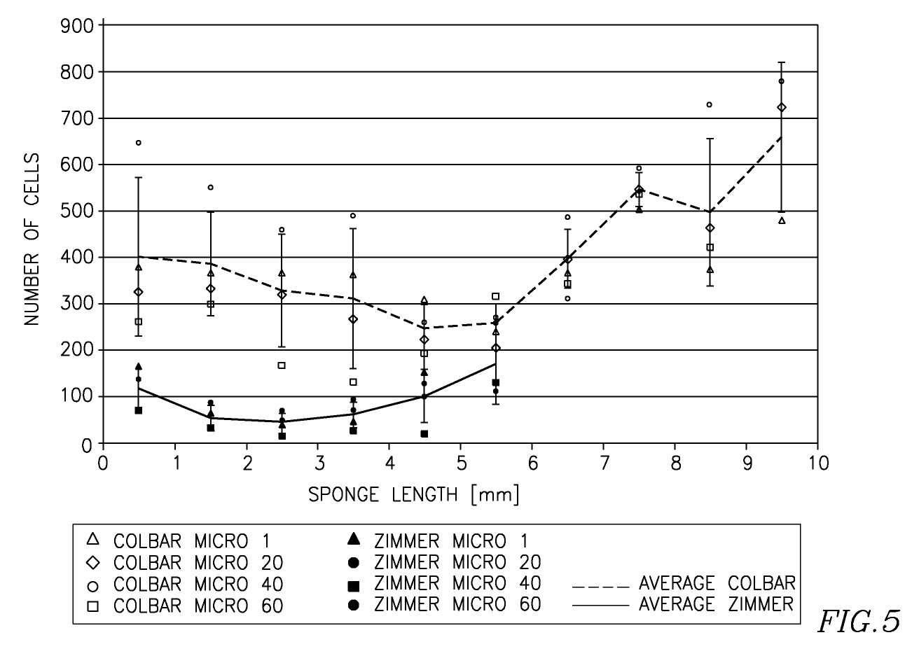

The cells were counted in each (1×1 mm) field automatically by the software. The stitched image size for the porcine ribose cross-linked collagen sponge was 15190×1976 pixels representing a section size of 10.5×1.1 millimeters. The stitched image size for the Sulzer sponge was 9091×1921 pixels representing a section size of 6.1×1.1 millimeters (note that the Sulzer sponge was shorter than the COLBAR porcine ribose cross-linked collagen sponge). For both sponges the area per count was 1×1 millimeters. The results of the automatic cell counting are illustrated in FIG. 5 below.

Reference is now made to FIG. 5 which is a schematic graph representing the results of an in-vitro experiment quantitatively comparing the fibroblast population of a collagen sponge based on ribose cross-linked porcine collagen with the fibroblast population of another commercially available collagen sponge based on collagen stabilized with formaldehyde.

In the graph of FIG. 5, the vertical axis represents the number of cells counted and the horizontal axis represents the length of the sponge in millimeters. The hollow symbols (hollow triangles, hollow rhomboids, hollow circles and hollow squares) represent the four different results of sections 1, 4, 7 and 10 taken at 1 microns 20 microns, 40 microns and 60 microns along the width of the sponge (in a direction perpendicular to the length and to the height of the sponge), respectively of the COLBAR reducing sugar cross-linked sponge. The dashed line associated with the hollow symbols represents a curve passing through the averaged value of the four cell counts (obtained from respective 1×1 millimeter fields of the first, fourth, seventh and tenth sections taken at each particular value of sponge length). The error bars represent the standard deviation of the mean for each averaged value of a group of four measurements at the specified sponge length.

The filled symbols (filled triangles, filled rhomboids, filled circles and filled squares) represent the four different results of sections 1, 4, 7 and 10 taken at 1 microns 20 microns, 40 microns and 60 microns along the width of the sponge (in a direction perpendicular to the length and to the height of the sponge), respectively of the Sulzer formaldehyde stabilized CollaCote® bovine collagen sponge. The continuous line associated with the filled symbols represents a curve passing through the averaged value of the four cell counts (obtained from respective 1×1 millimeter fields of the first, fourth, seventh and tenth sections taken at each particular value of Sulzer sponge length). The error bars represent the standard deviation of the mean for each averaged value of a group of four measurements at the specified sponge length.

It may be seen from the graph of FIG. 5 that the averaged cell counts are consistently significantly higher in the COLBAR sponge than in the Sulzer sponge. In both sponges, the cell count is higher towards the end of the sponge than in the middle portion of the sponge which may possible (but not necessarily) be due to effects associated with the rate of migration of fibroblasts from the sponge's edge to the inner part of the sponge.

It is further noted that for the COLBAR sponge, the cell count near one edge along the length of the sponge (represented by the value of 0.5 millimeters on the horizontal axis) is significantly higher than the cell count at the opposite edge of the same sponge (represented by the value of 9.5 millimeters on the horizontal axis). This may be possibly attributed to the higher density of the sponge at 9.5 millimeter end of the sponge because this end of the sponge was in contact with the lyophylization tray bottom during the lyophilization of the sponge resulting in denser (and probably less penetrable) sponge structure at this end of the COLBAR sponge.

However, it is noted that the cell counts of the COLBAR sponge are always higher than the cell counts of the Sulzer sponge at the corresponding length. The increase in cell count ranges from a cell count increase of about 358% in the cell count of the COLBAR sponge relative to the Sulzer sponge at 0.5 millimeter sponge length, to a cell count increase of about 565% in the cell count at the center of the COLBAR sponge (at 4.5 millimeters sponge length) relative to the center of the Sulzer sponge (at 2.5 millimeter sponge length).

If one compares the peak value (at the 9.5 millimeter length) of the COLBAR sponge with the peak value (at the 5.5 millimeter length) of the Sulzer sponge, the cell count increase of the COLBAR sponge relative to the Sulzer sponge is about 389%.

It may be concluded that in comparison to Sulzer CollaCot® sponge, the COLBAR ribose cross-linked porcine collagen sponge produced as disclosed hereinabove is substantially and unexpectedly more conducive to penetration, growth and proliferation of primary human fibroblast cultured under the same conditions.

It is noted that while the reasons for this advantage of the COLBAR sponge are not clear at the present, it may possibly be due to the fact that small amounts of the cross-linker may be slowly released from the cross-linked collagen of both sponges. While the nature and chemical composition of any such substances released from a reducing sugar cross-linked collagen is not clearly known or characterized (due to possible secondary rearrangement of the cross-links of the glycated collagen), it is a well documented fact that small amounts of formaldehyde may actually retard or inhibit cell proliferation due to their toxicity.

It may also be possible (but not proven herein) that the actual structure and moieties presented to cells by the glycated and/or reducing sugar cross-linked collagen matrix itself is more favorable to or supportive of cell migration and/or penetration, and/or viability and/or proliferation than the structure or moieties presented by the Sulzer collagen sponge and/or other non-glycated, cross-linked collagen matrices.

It is noted that while the experiment of EXAMPLE 1 described above demonstrates the implementation of the composite matrix based on the use of a combination of a reducing sugar cross-linked lower density collagen scaffold and a higher density membrane-like barrier comprising compressed reducing sugar cross-linked collagen, this is by way of example only and is not intended to limit the composition of the composite matrix of the present application to reducing sugar cross-linked collagen material only. Rather, additional types of materials may be added to the matrices of the composite matrix.

For example, the portions 16 and/or 18 of the composite matrix 14, and the portions 2 and/or 4 of the matrix 1 of FIG. 2 may also include, in addition to the reducing sugar cross-linked collagen, other types of biocompatible materials or any suitable mixtures of biocompatible materials for modifying the properties of the matrices or of a selected portion of the device. Such materials may include but are not limited to, hyaluronic acid (HA) and/or hyaluronan and/or suitable derivatives and/or salts and/or esters thereof, chitosan and/or hyaluronan and/or suitable derivatives and/or salts and/or esters thereof, various oligosaccharides and/or polysaccharides and/or suitable derivatives and/or salts and/or esters thereof, various biocompatible synthetic polymers as is known in the art, cross-linked and/or non-cross-linked proteins (such as, but not limited to, alkaline phosphatase and/or pyrophosphatase which play a role in mineralization of new bone), cross-linked and/or non-cross-linked glycoproteins and the like, calcium phosphate nano-particles and/or hydroxy-apatite crystals (which may be used to accelerate bone augmentation), growth factors such as, but not limited to BMP's, PDGF and the like, including any growth factors known in the art.), any suitable combinations of the above may also be used

It is noted that in accordance with an embodiment of the invention it may be possible to add additional substances and additives to the composite membranes described either before or after the cross-linking of the membrane.

Additionally, the materials or substances that may be added to the composite membranes of the present invention are not limited to structural materials such as natural and/or synthetic polymers and the like but may also include other types of additives, including but not limited to, small molecules, drugs, anesthetic material(s), analgesic material(s) or any other desired material or substance. Any combinations of the above materials with any other materials disclosed in the present application may also be used.

The additional materials added to the reducing sugar cross-linked collagen forming the implanted matrices of the present invention may be cross linked or non-cross-linked, biocompatible, natural or synthetic polymers. Such polymers or other substances which may be added to the collagen-based matrices of the implants of the invention may be trapped within and/or cross-linked to the collagen during the glycation and/or cross-linking process used to form the composite matrix as described in Examples 1-3 above.

For example, if chitosan is used as an additive to one or more of the portions 2 and 4 of the matrices of the device 4, the glycation process and subsequent cross-linking cross-links not only the molecules of collagen to each other but also forms cross-links attaching the chitosan backbone to collagen molecules through the glycation of free amino groups in chitosan and the lysine amino groups in collagen. The resulting composite matrix may have different, biological and physico-chemical characteristics. Co-pending U.S. provisional application Ser. No. 60/713,390 to Bayer et al., filed Sep. 2, 2005 discloses, inter alia, such cross-linked matrices including collagen and amino-group containing polysachharides or amino derivatized polysaccharides and methods for their preparation.

It is further noted that while the glycation and cross-linking reactions used to form the reducing sugar cross-linked collagen matrices of the composite matrix described in EXAMPLE 1 makes use of DL-glyceraldehyde as the cross-linking reducing sugar, any other cross-linking reducing sugar or reducing sugar derivatives known in the art may be used for cross-linking of the collagen matrices forming the composite matrices of the present invention. For example, cross-linking in aqueous solutions is described in U.S. Pat. Nos. 5,955,438 and 6,346,515 to Pitaru et al., which are both incorporated herein by reference in their entirety. The methods, cross-linking reducing sugars and collagen types described in these patents may all be used in making the composite matrices and devices of the present invention. Similarly, all the methods, cross-linking sugars, solvent systems (including polar or hydrophilic solvents and water with or without suitable buffers and/or salts) and collagen types described in U.S. Pat. No. 6,682,760 to Noff et al., incorporated herein by reference in its entirety may also be used for preparing and cross-linking the composite matrices and devices of the present invention.

It is also noted that the cross-linking methods used in the cross-linking of the embodiments of the composite multi-density membranes of the present invention may be applied using either D or L forms or mixtures of D and L forms of reducing sugars or reducing sugar derivatives, as is known in the art.

Methods for preparing mixed matrices of collagen and various amino group containing polysaccharides and/or amino derivatized polysaccharides are described in co-pending U.S. provisional patent application Ser. No. 60/713,390 application to Bayer et al., filed on Sep. 2, 2005, entitled “CROSS-LINKED POLYSACCHARIDE MATRICES AND METHODS FOR THEIR PREPARATION” incorporated herein by reference. The methods, materials and derivatizing reaction described in co-pending provisional application Ser. No. 60/713,390 may also be adapted and/or used for preparing mixed type composite matrices in accordance with an additional embodiment of the present invention.

It is further noted that while the examples of the composite matrices disclosed hereinabove have two portions or layers each having a different collagen density, the composite matrices of the invention may have more then two layers or more then two portions. For example, in accordance with yet another embodiment of the present invention, a composite matrix having three portions may be made and used for bone induction or conduction. This may be accomplished by adding an additional layer of fibrillated collagen having a low density of collagen particles on top of the portion 2 of the implant 1 before drying to for a three layer composite matrix having three portions each having a different density of collagen. The three layered composite matrix may then be dried and cross-linked using a reducing sugar in a reaction mixture with or without a polar solvent as described hereinabove. The resulting three layered composite matrix may then be washed and dried or lyophilized as described hereinabove.

It is further noted that the size and shape of the composite matrix having two or more layers of glycated reducing sugar cross-linked collagen may vary according to need and type of bone defect in need of treatment. Thus the thickness of the various layers or portions of the implanted matrix may be varied at will by controlling the amount and/or the concentration of material used when forming each layer or portion of the matrix. Any type of shape, size, number of layers or portions and the thickness of each layer or portion may be used in the matrices of the present invention, depending, inter alia, on the specific application.

It will be appreciated by those skilled in the art that it may also be possible, in accordance with another embodiment of the present invention to make matrices having a density gradient along one or more dimensions of the portion of the matrix or along the entire span of the matrix. Various different methods for forming density gradients within one or more of the portions of a matrix may be used. For example one may use centrifugation techniques to form a density gradient along a dimension of one or more of the portions 2 and 4 of the matrix 1 of FIG. 2. Other methods for forming continuous or discontinuous density gradients may include, but are not limited to, mixing of two different suspensions each having a different density of collagen based material therein and overlaying of the resulting mixture on top of the layer 4. However, any other method for gradient forming known in the art, such as but not limited to spinning method, may be used in forming composite matrices having density gradients.

It is further noted that in accordance with yet another embodiment of the present invention, it may be useful to include in the composite matrices of the present invention various different added materials or additives which may be incorporated into the matrix to be released later. Such additives may include, but are not limited to, relatively small or intermediate size molecules materials or substances such as, but not limited to, antimicrobial agent(s), an anti-inflammatory agent(s), anti-bacterial agent(s), anti-fungal agent(s), one or more factors having tissue inductive properties, growth factors, growth promoting and/or growth inhibiting proteins or factors, extracellular matrix components, anesthetic material(s), analgesic material(s), BMP's, osteoblast attracting factors or substances, and any other desired drugs or pharmaceutical agent(s) or compositions.

Other substances or compounds which may be included in the composite matrices of the present may include, inter alia, various proteins, glycoproteins, mucoproteins, mucopolysaccharides, glycosaminoglycans such as but not limited to chondroitin 4-sulfate, chondroitin 6-sulfate, keratan sulfate, dermatan sulfate, heparin, heparan sulfate, hyaluronan, proteoglycans such as the lecitin rich interstitial proteoglycans decorin, biglycan, fibromodulin, lumican, aggrecan, syndecans, beta-glycan, versican, centroglycan, serglycin, fibronectins, fibroglycan, chondroadherins, fibulins, thrombospondin-5, calcium phosphate, hydroxyapatite, alkaline phosphatase and pyrophosphatase.

In addition any material(s) related to gene therapy may also be included in the composite matrices of the present invention, such as, but not limited to, DNA, RNA, fragments of DNA or RNA, nucleic acids, oligonucleotides, polynucleotides, anti-sense DNA or RNA, plasmids, vectors or the like, allogeneic material(s) a nucleic acid, an oligonucleotide, a chimeric DNA/RNA construct, DNA or RNA probes, anti-sense DNA, anti-sense RNA, a gene, a part of a gene, a composition including naturally or artificially produced oligonucleotides, a plasmid DNA, a cosmid DNA, modified viral genetic constructs or any other substance or compound containing nucleic acids or chemically modified nucleic acids, or various combinations or mixtures of the above disclosed substances, compounds and genetic constructs, and may also include the vectors required for promoting cellular uptake and transcription, such as but not limited to various viral or non-viral vectors known in the art.

It is noted that any combinations of any of the substances, materials, additives, genetic constructs, gene therapy materials, drugs, and any other additives disclosed hereinabove and/or hereinafter may be added to the composite matrices of the present application.

All the above disclosed materials or substances and any combinations of such materials or substances which may be used as additives to the composite membranes of the present invention may be added either before or after the performing of the cross-linking reaction (using the reducing sugar cross-linker). However, it may also be possible to add one or more additives, perform the cross-linking of the collagen and then add additional substance(s) by soaking the cross-linked collagen in a solution including one or more additional substances and/or additives.

It will be appreciated by those skilled in the art that the implantable devices and/or composite membranes of the present invention may also be modified by the inclusion of living cells. Such living cells may be autologous cells derived from the patient in which the implant is going to be implanted but may also be cells from a genetically compatible donor. The cells may be any type of living cells which may have a supporting role or assisting role in bone formation, such as but not limited to osteoblasts, progenitor cells, stem cells, precursor cells, embryonic stem cells, adult derived stem cells, cells derived from cell cultures or cell lines, non-differentiated cells, or the like. Such cells may be added to the devices of the present invention by soaking the devices or implants or parts thereof in suspensions of such cells or in culture medium in which such cells are present. Alternatively, the implant, device or composite membranes may be incubated together with any of the above disclosed cells for a sufficient period of time to ensure penetration or migration of such cells into the scaffold part of the device or composite membranes. After the incubation or other cell addition procedures the devices, implants or composite membranes charged with cells may be implanted in or inserted into the bone defect as described hereinabove.

Such additives or materials may be simply mixed with the collagen based material used for preparation of the composite matrices before the cross-linking step. After the collagen and/or compositions containing collagen mixed with other polymers are cross-linked some or all of the added substances or additives may be trapped in the cross-linked matrix (or matrices) and may be released from the matrix to exert their biological influence within or in the vicinity of the defect. Alternatively, some molecules containing amino groups (such as, but not limited to, lysine or arginine containing proteins and polypeptides, and the like) may be covalently linked to the collagen or polysaccharide backbones through collagen (lysine) amino groups or through amino groups of the polysaccharide used in mixed membranes by the glycation reactions and further rearrangement and/or cross-linking steps. Such covalently linked molecules or agents may modify the structure and physiological properties of the resulting matrices and may confer various useful biological properties thereon, as is known in the art, such as, for example, serving as molecular cues for cells which penetrate the scaffold, etc.

It is further noted that the composite matrices of the invention as described hereinabove may also be seeded prior to implantation thereof with any suitable type of living cells which may be useful for assisting or improving bone tissue formation within the matrix or the bone defect, such cells may include but are not limited to, osteoblasts, stem cells, or any other bone building cells known in the art.

It is noted that any type of collagen may be used in the composite matrices of the present invention including but not limited to, native collagen, fibrillar collagen, fibrillar atelopeptide collagen, lyophylized collagen, collagen obtained from animal sources, human collagen, recombinant collagen, proteolitically digested collagen, pepsinized collagen, reconstituted collagen, collagen types I, II and IX, or any other suitable mixture of any other types of collagen known in the art and any combinations thereof.

It is noted that for the purpose of the present application the words “glycated collagen” mean any type of collagen which was reacted with a reducing sugar or with a reducing sugar derivative and also include all types of cross-linked collagen which may be formed in subsequent rearrangement and/or cross-linking following the glycation of the collagen.

It will be appreciated by those skilled in the art that while the examples disclosed hereinabove are described with respect to alveolar bone augmentation, the devices and methods described herein are not limited to oral surgical procedures described and may be easily modified and adapted for any type of procedure involving treatment of bone defects, fractures, and the like in any type of bone for orthopedic, plastic, cosmetic and other types of surgery and bone graft implant procedures. Thus the composite matrices of the invention may be used to treat any type of bone defect or bone fracture of any type of bones in humans or other species of animals.

It is noted that any of the composite glycated collagen based and/or reducing sugar cross-linked collagen based implants disclosed herein and any of the reducing sugar cross-linked collagen based scaffolds and sponges disclosed in the present application may also include one or more additives such as but not limited to, an antimicrobial agent, an anti-inflammatory agent, an anti-bacterial agent, an anti-fungal agent, one or more factors having tissue inductive properties, growth factors, growth promoting and/or growth inhibiting proteins or factors, extracellular matrix components, an anesthetic material, an analgesic material, an osteoblast attracting factor, a drug, a pharmaceutical agent, a pharmaceutical composition, a protein, a glycoprotein, a mucoprotein, a mucopolysaccharide, a glycosaminoglycan, hyaluronic acid, chondroitin 4-sulfate, chondroitin 6-sulfate, keratan sulfate, dermatan sulfate, heparin, heparan sulfate, a proteoglycan, a lecitin rich interstitial proteoglycan, decorin, biglycan, fibromodulin, lumican, aggrecan, syndecans, beta-glycan, versican, centroglycan, serglycin, fibronectins, fibroglycan, chondroadherins, fibulins, thrombospondin-5, calcium phosphate, hydroxyapatite, alkaline phosphatase, pyrophosphatase, a material related to gene therapy, DNA, RNA, a fragment of DNA or RNA, a nucleic acid, an oligonucleotide, a polynucleotide, a plasmid, a vector, an allogeneic material, a nucleic acid, an oligonucleotide, a chimeric DNA/RNA construct, a DNA probe, an RNA probe, anti-sense DNA, anti-sense RNA, a gene, a part of a gene, a composition including naturally or artificially produced oligonucleotides, a plasmid DNA, a cosmid DNA, a viral genetic construct, hyaluronan, a hyaluronan derivative, a hyaluronan salt a hyaluronan ester, chitosan, a chitosan derivative, a chitosan salt, a chitosan ester thereof, an oligosaccharide, a polysaccharides, a polysaccharides salt, a polysaccharides derivative, a polysaccharides ester, an oligosaccharide derivative, an oligosaccharide salt, an oligosaccharide ester, a biocompatible synthetic polymer, a cross-linked protein, a cross-linked glycoprotein, a non-cross-linked glycoprotein, calcium phosphate nanoparticles, hydroxy-apatite crystals, a growth factors, a BMP, PDGF and any combinations thereof.

Additionally, any of the composite and/or reducing sugar cross-linked collagen based implants disclosed herein and any of the glycated collagen based and/or reducing sugar cross-linked collagen based scaffolds and sponges disclosed in the present application may also include living cells therein. The living cells may include but are not limited to cultured cells, stem cells, human cells, animal cells, fibroblasts, pluripotent bone marrow cells, pluripotent stem cells, bone building cells, osteoblasts, mesenchymal cells, mammalian cells, primary cells, genetically modified cells, nerve cells and any combinations thereof. Such cells may be introduced into the composite implants and/or sponges and or scaffolds by suitable seeding and incubation, as disclosed hereinabove or by any other method for cell seeding known in the art.

Moreover, while the specific examples of the composite sponges, implants and the scaffold materials disclosed herein are glycated and cross-linked using a single reducing sugar, this is by no means obligatory and any of the above disclosed composite sponges, implants and scaffold materials may also be glycated and/or cross-linked by using any suitable mixture of the reducing sugars disclosed hereinabove. Similarly, while the specific examples of the composite sponges, implants and the scaffold materials disclosed herein are made by glycation and/or and cross-linking of a single type of collagen, this is not obligatory and any of the above disclosed collagen types including also any suitable mixture of different collagen types (with or without additives and/or additional polymers, and/or living cells) may be used in making the composite sponges, implants and scaffold materials disclosed hereinabove.

What is claimed is:

1. A method for using a reducing sugar cross-linked collagen matrix as an improved scaffold for cell proliferation and cell differentiation, the method comprising the steps of:
providing a scaffold comprising a collagen matrix cross-linked with a reducing sugar; and
incubating said scaffold with living cells to induce improved growth and/or proliferation and/or differentiation of said cells.

2. The method according to claim 1 wherein said cells are selected from cultured cells, stem cells, human cells, animal cells, fibroblasts, pluripotent bone marrow cells, pluripotent stem cells, bone building cells, osteoblasts, mesenchymal cells, mammalian cells, primary cells, genetically modified cells, nerve cells and any combinations thereof.

3. The method according to claim 1 wherein said scaffold is obtained by incubating a collagen based matrix with a reducing sugar in an incubation solution comprising ethanol.

4. The method according to claim 3 wherein said incubation solution comprises 70% ethanol.

5. The method according to claim 1 wherein said reducing sugar is selected from D(-) ribose and DL glyceraldehyde.

6. The method according to claim 1 wherein said scaffold comprises at least one additional substance.

7. The method according to claim 6 wherein said at least one additional substance is selected from an antimicrobial agent, an anti-inflammatory agent, an anti-bacterial agent, an anti-fungal agent, one or more factors having tissue inductive properties, growth factors, growth promoting and/or growth inhibiting proteins or factors, extracellular matrix components, an anesthetic material, an analgesic material, an osteoblast attracting factor, a drug, a pharmaceutical agent, a pharmaceutical composition, a protein, a glycoprotein, a mucoprotein, a mucopolysaccharide, a glycosaminoglycan, hyaluronic acid, chondroitin 4-sulfate, chondroitin 6-sulfate, keratan sulfate, dermatan sulfate, heparin, heparan sulfate, a proteoglycan, a lecitin rich interstitial proteoglycan, decorin, biglycan, fibromodulin, lumican, aggrecan, syndecans, beta-glycan, versican, centroglycan, serglycin, fibronectins, fibroglycan, chondroadherins, fibulins, thrombospondin-5, calcium phosphate, hydroxyapatite, alkaline phosphatase, pyrophosphatase, a material related to gene therapy, DNA, RNA, a fragment of DNA or RNA, a nucleic acid, an oligonucleotide, a polynucleotide, a plasmid, a vector, an allogeneic material, a nucleic acid, an oligonucleotide, a chimeric DNA/RNA construct, a DNA probe, an RNA probe, anti-sense DNA, anti-sense RNA, a gene, a part of a gene, a composition including naturally or artificially produced oligonucleotides, a plasmid DNA, a cosmid DNA, a viral genetic construct, hyaluronan, a hyaluronan derivative, a hyaluronan salt a hyaluronan ester, chitosan, a chitosan derivative, a chitosan salt, a chitosan ester thereof, an oligosaccharide, a polysaccharides, a polysaccharides salt, a polysaccharides derivative, a polysaccharides ester, an oligosaccharide derivative, an oligosaccharide salt, an oligosaccharide ester, a biocompatible synthetic polymer, a cross-linked protein, a cross-linked glycoprotein, a non-cross-linked glycoprotein, calcium phosphate nanoparticles, hydroxy-apatite crystals, a growth factors, a BMP, PDGF and any combinations thereof.

8. The method according to claim 1 wherein said step of incubating comprises incubating said scaffold with living cells in vivo to induce improved growth and/or proliferation and/or differentiation of said cells.

9. The method according to claim 1 wherein said step of incubating comprises incubating said scaffold with living cells in vitro to induce improved growth and/or proliferation and/or differentiation of said cells.
